(19) United States
(12) Patent Application Publication
(10) Pub. No.: US 2024/0342462 A1
(43) Pub. Date: Oct. 17, 2024

(54) METHODS AND SYSTEMS FOR DETERMINING POSITIONING OF A HEART PUMP

(71) Applicant: Abiomed, Inc., Danvers, MA (US)

(72) Inventors: Mohammed Alwatban, Danvers, MA (US); Ahmad El Katerji, Danvers, MA (US); Sanket Yograj Thakare, Danvers, MA (US); Atahar Mostafa, Danvers, MA (US); Yuting Zhang, Danvers, MA (US)

(73) Assignee: Abiomed, Inc., Danvers, MA (US)

(21) Appl. No.: 18/621,649

(22) Filed: Mar. 29, 2024

Related U.S. Application Data
(60) Provisional application No. 63/493,505, filed on Mar. 31, 2023.

(51) Int. Cl. A61M 60/531; A61M 60/508

(52) U.S. Cl. CPC A61M 60/531; A61M 60/508; A61M 2205/3334

(57) ABSTRACT

Methods and apparatus for determining a position of a heart pump in a heart of a patient are described. The method includes receiving a pressure signal from at least one pressure sensor arranged on the heart pump, generating a histogram of values observed within a time window associated with the pressure signal, and determining the position of the heart pump based, at least in part, on a morphology of the histogram.

### CROSS REFERENCE TO RELATED APPLICATIONS

This application claims the benefit under 35 U.S.C. § 119(e) to U.S. Provisional Patent Application No. 63/493,505, filed Mar. 31, 2023, and titled, “METHODS AND SYSTEMS FOR DETERMINING POSITIONING OF A HEART PUMP,” the entire contents of which is incorporated herein by reference in its entirety.

### FIELD OF THE INVENTION

This disclosure relates to techniques for determining the positioning of a heart pump.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span one or more heart valves. The intravascular blood pump is typically disposed at the end of the catheter. Once in position, the pump may be used to assist the heart and pump blood through the circulatory system and, therefore, temporarily reduce load on the patient's heart, such as to enable the heart to recover after a heart attack. An exemplary intravascular blood pump is available from ABIOMED, Inc., Danvers, MA under the tradename Impella® heart pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. In some instances, the controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

An intravascular blood pump designed for right heart assistance can extend through the pulmonary valve and into the pulmonary artery in order to expel blood into the pulmonary artery. To properly position certain right heart assistance devices, the device may be passed through the inferior vena cava, right atrium, tricuspid valve, right ventricle and finally the pulmonary valve. Proper positioning of the intravascular blood pump across the pulmonary valve is important to ensure the pump operates as intended. Described herein are systems and methods for determining a position of an intravascular blood pump based, at least in part, on an analysis of a pressure signal received from a pressure sensor located on the pump. Although the techniques described herein are used to determine the position of a blood pump configured to provide right heart support, it should be appreciated that at least some of the techniques may also be used to determine the position of a blood pump inserted across the aortic valve in the left side of the heart.

In one aspect, a method of determining a position of a heart pump in a heart of a patient is provided. The method includes receiving a pressure signal from at least one pressure sensor arranged on the heart pump, generating a histogram of values observed within a time window associated with the pressure signal, and determining the position of the heart pump based, at least in part, on a morphology of the histogram.

In another aspect, the at least one pressure sensor comprises a differential pressure sensor. In another aspect, a length of the time window is at least four seconds and less than ten seconds. In another aspect, the length of the time window is at least five seconds and less than seven seconds.

In another aspect, the method further includes filtering the pressure signal to generate a filtered pressure signal, and generating a histogram of values includes generating a histogram of values observed within a time window of the filtered pressure signal. In another aspect, filtering the pressure signal comprises filtering the pressure signal with a finite impulse response (FIR) filter.

In another aspect, determining the position of the heart pump based, at least in part, on a morphology of the histogram includes determining the position of the heart pump based, at least in part, on a distribution of the histogram. In another aspect, determining the position of the heart pump based, at least in part, on a distribution of the histogram includes determining whether the distribution is a bimodal distribution. In another aspect, determining the position of the heart pump based, at least in part, on a distribution of the histogram further includes determining that the heart pump is not positioned properly when the histogram has the bimodal distribution. In another aspect, determining the position of the heart pump based, at least in part, on a distribution of the histogram further includes determining whether the distribution is a normal distribution. In another aspect, determining the position of the heart pump based, at least in part, on a distribution of the histogram further includes determining that the heart pump is positioned properly when the histogram has the normal distribution.

In another aspect, determining the position of the heart pump based, at least in part, on a morphology of the histogram includes determining a standard deviation of values within the time window, defining a sub-window within the time window based, at least in part, on the standard deviation, and determining the position of the heart pump based, at least in part, on the values within the sub-window. In another aspect, determining the position of the heart pump based, at least in part, on the values in the sub-window includes calculating a first sum of all values within the sub-window, calculating a second sum of all values within the time window, dividing the first sum by the second sum to determine a morphology index value, determining that the heart pump is not positioned properly when the morphology index value is less than a threshold value, and determining that the heart pump is positioned properly when the morphology index value is greater than the threshold value.

In another aspect, the method further includes outputting via a user interface, an indication of the position of the heart pump. In another aspect, the method further includes determining a pulsatility of the pressure signal, and generating a histogram of values is performed in response to the pulsatility of the pressure signal being above a first threshold pulsatility value and below a second threshold pulsatility value. In another aspect, the heart pump is configured to provide right heart support for the patient, and determining the position of the heart pump based, at least in part, on a morphology of the histogram comprises determining whether an outlet of the heart pump is located in a pulmonary artery of the patient.

In one aspect, a heart pump system is provided. The heart pump system includes a heart pump including at least one pressure sensor configured to sense a pressure within a portion of a heart of a patient. and a controller. The controller is configured to receive a pressure signal output from the at least one pressure sensor, generate a histogram of values observed within a time window associated with the pressure signal, and determine the position of the heart pump based, at least in part, on a morphology of the histogram.

In another aspect, the at least one pressure sensor comprises a differential pressure sensor. In another aspect, a length of the time window is at least four seconds and less than ten seconds. In another aspect, the length of the time window is at least five seconds and less than seven seconds.

In another aspect, the controller is further configured to filter the pressure signal to generate a filtered pressure signal, and generating a histogram of values includes generating a histogram of values observed within a time window of the filtered pressure signal. In another aspect, filtering the pressure signal includes filtering the pressure signal with a finite impulse response (FIR) filter.

In another aspect, determining the position of the heart pump based, at least in part, on a morphology of the histogram includes determining the position of the heart pump based, at least in part, on a distribution of the histogram. In another aspect, determining the position of the heart pump based, at least in part, on a distribution of the histogram includes determining whether the distribution is a bimodal distribution. In another aspect, determining the position of the heart pump based, at least in part, on a distribution of the histogram further includes determining that the heart pump is not positioned properly when the histogram has the bimodal distribution. In another aspect, determining the position of the heart pump based, at least in part, on a distribution of the histogram further comprises determining whether the distribution is a normal distribution. In another aspect, determining the position of the heart pump based, at least in part, on a distribution of the histogram further includes determining that the heart pump is positioned properly when the histogram has the normal distribution.

In another aspect, determining the position of the heart pump based, at least in part, on a morphology of the histogram includes determining a standard deviation of values within the time window, defining a sub-window within the time window based, at least in part, on the standard deviation, and determining the position of the heart pump based, at least in part, on the values within the sub-window. In another aspect, determining the position of the heart pump based, at least in part, on the values in the sub-window includes calculating a first sum of all values within the sub-window, calculating a second sum of all values within the time window, dividing the first sum by the second sum to determine a morphology index value, determining that the heart pump is not positioned properly when the morphology index value is less than a threshold value, and determining that the heart pump is positioned properly when the morphology index value is greater than the threshold value.

In another aspect, the controller is further configured to output via a user interface, an indication of the position of the heart pump. In another aspect, the controller is further configured to determine a pulsatility of the pressure signal, and generating a histogram of values is performed in response to the pulsatility of the pressure signal being above a first threshold pulsatility value and below a second threshold pulsatility value. In another aspect, the heart pump is configured to provide right heart support for the patient, and determining the position of the heart pump based, at least in part, on a morphology of the histogram comprises determining whether an outlet of the heart pump is located in a pulmonary artery of the patient.

In one aspect, a controller for a heart pump system is provided. The controller includes at least one hardware processor. The at least one hardware processor is configured to receive a pressure signal output from at least one pressure sensor arranged on a heart pump of the heart pump system, generate a histogram of values observed within a time window associated with the pressure signal, and determine the position of the heart pump based, at least in part, on a morphology of the histogram.

In another aspect, the at least one pressure sensor comprises a differential pressure sensor. In another aspect, a length of the time window is at least four seconds and less than ten seconds. In another aspect, the length of the time window is at least five seconds and less than seven seconds.

In another aspect, the at least one hardware processor is further configured to filter the pressure signal to generate a filtered pressure signal, and generating a histogram of values comprises generating a histogram of values observed within a time window of the filtered pressure signal. In another aspect, filtering the pressure signal includes filtering the pressure signal with a finite impulse response (FIR) filter.

In another aspect, determining the position of the heart pump based, at least in part, on a morphology of the histogram includes determining the position of the heart pump based, at least in part, on a distribution of the histogram. In another aspect, determining the position of the heart pump based, at least in part, on a distribution of the histogram includes determining whether the distribution is a bimodal distribution. In another aspect, determining the position of the heart pump based, at least in part, on a distribution of the histogram further includes determining that the heart pump is not positioned properly when the histogram has the bimodal distribution. In another aspect, determining the position of the heart pump based, at least in part, on a distribution of the histogram further comprises determining whether the distribution is a normal distribution. In another aspect, determining the position of the heart pump based, at least in part, on a distribution of the histogram further includes determining that the heart pump is positioned properly when the histogram has the normal distribution.

In another aspect, determining the position of the heart pump based, at least in part, on a morphology of the histogram includes determining a standard deviation of values within the time window, defining a sub-window within the time window based, at least in part, on the standard deviation, and determining the position of the heart pump based, at least in part, on the values within the sub-window. In another aspect, determining the position of the heart pump based, at least in part, on the values in the sub-window includes calculating a first sum of all values within the sub-window, calculating a second sum of all values within the time window, dividing the first sum by the second sum to determine a morphology index value, determining that the heart pump is not positioned properly when the morphology index value is less than a threshold value, and determining that the heart pump is positioned properly when the morphology index value is greater than the threshold value.

In another aspect, the at least one hardware processor is further configured to output via a user interface, an indication of the position of the heart pump. In another aspect, the at least one hardware processor is further configured to determine a pulsatility of the pressure signal, and generating a histogram of values is performed in response to the pulsatility of the pressure signal being above a first threshold pulsatility value and below a second threshold pulsatility value. In another aspect, the heart pump is configured to provide right heart support for a patient, and determining the position of the heart pump based, at least in part, on a morphology of the histogram includes determining whether an outlet of the heart pump is located in a pulmonary artery of the patient.

In one aspect, a method of determining a position of a right heart cardiac support device in a heart of a patient is provided. The method includes receiving a pressure signal from at least one pressure sensor arranged adjacent to an outlet of the right heart cardiac support device, generating a histogram of values observed within a time window associated with the pressure signal, determining that a distribution of the histogram is bimodal, and outputting an indication that an outlet of the right heart cardiac support device is not located with a pulmonary artery of the patient in response to determining that the distribution of the histogram is bimodal.

In another aspect, the at least one pressure sensor comprises a differential pressure sensor. In another aspect, a length of the time window is at least four seconds and less than ten seconds. In another aspect, the length of the time window is at least five seconds and less than seven seconds.

In another aspect, the method further includes filtering the pressure signal to generate a filtered pressure signal, and generating a histogram of values comprises generating a histogram of values observed within a time window of the filtered pressure signal. In another aspect, filtering the pressure signal comprises filtering the pressure signal with a finite impulse response (FIR) filter.

In another aspect, determining that the distribution of the histogram is bimodal includes determining a standard deviation of values within the time window, defining a sub-window within the time window based, at least in part, on the standard deviation, and determining that the distribution of the histogram is bimodal based, at least in part, on the values within the sub-window. In another aspect, determining that the distribution of the histogram is bimodal based, at least in part, on the values within the sub-window includes calculating a first sum of all values within the sub-window, calculating a second sum of all values within the time window, dividing the first sum by the second sum to determine a morphology index value, and determining that distribution of the histogram is bimodal when the morphology index value is less than a threshold value.

In another aspect, the method further includes determining a pulsatility of the pressure signal, and generating a histogram of values is performed in response to the pulsatility of the pressure signal being above a first threshold pulsatility value and below a second threshold pulsatility value.

### BRIEF DESCRIPTION OF DRAWINGS

FIG. 1A shows an illustrative cardiac support device that may be used with some embodiments.

FIG. 1B shows an illustrative cardiac support system that includes the cardiac support device of FIG. 1A.

FIG. 2 is a flowchart of a process for determining a position of a cardiac support device, in accordance with some embodiments.

FIG. 3 is a flowchart of a process for determining a morphology index associated with a pressure signal waveform, in accordance with some embodiments.

FIG. 4 schematically illustrates a process for analyzing a morphology of a histogram associated with a pressure signa, in accordance with some embodiments.

### DETAILED DESCRIPTION

Determining that a cardiac support device (e.g., an intravascular blood pump) is properly positioned in the heart of a patient during its operation may be important to ensure the pump is providing adequate cardiac support to the patient. As described herein, the cardiac support device may include one or more pressure sensors configured to sense a pressure within a patient's heart as the device operates. For example, the pressure sensor(s) may include an optical pressure sensor and/or a differential pressure sensor configured to sense a pressure difference across one or more valves through which the cardiac support device is inserted. The positioning of a cardiac support device inserted across the aortic valve to provide left heart support for a patient may be determined, at least in part, by evaluating the pulsatility of the pressure signal sensed by the one or more pressure sensors on the device. The inventors have recognized and appreciated that, in some instances, the pulsatility metric used to determine positioning of a left heart support device may not be used to provide a reliable positioning determination when the cardiac support device is used to provide right heart support. For instance, because pumping of blood through the right side of the heart generally produces a pressure signal with smaller pulsatility as compared to the pulsatility of a pressure signal measured when blood is pumped through the left side of the heart, determining the positioning of a right heart support system based solely or primarily on the pulsatility of the pressure signal may not lead to an accurate positioning result. To this end, some embodiments of the present disclosure relate to novel techniques for determining the position of a cardiac support system (e.g., a right heart cardiac support system) based, at least in part, on a morphology index associated with the pressure signal.

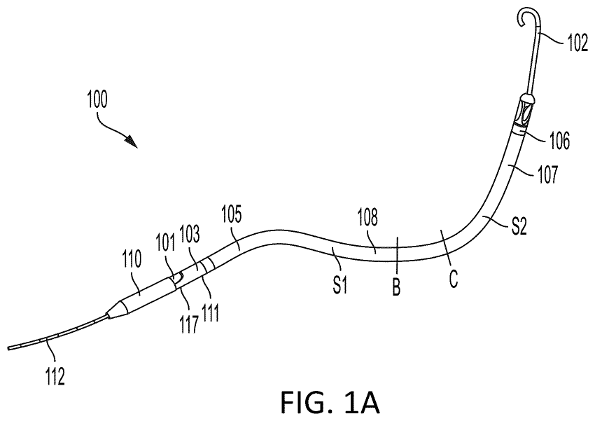

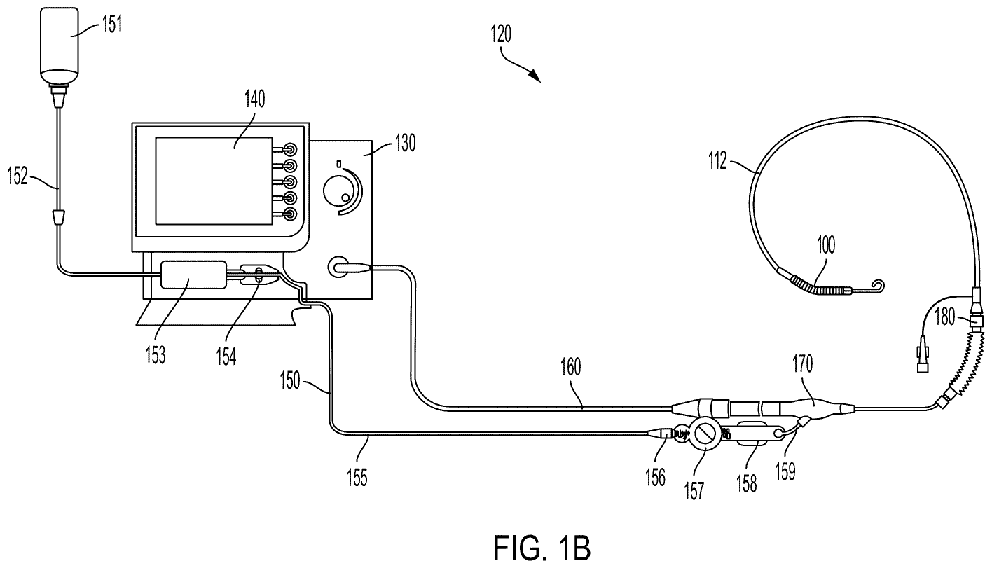

FIG. 1A shows an illustrative embodiment of a blood pump assembly 100 according to the present disclosure. The blood pump assembly 100 may include a pump 101, a pump housing 103, a proximal end 105, a distal end 107, a cannula 108, an impeller (not shown), an atraumatic extension 102, a catheter 112, an inlet area 110, an outlet area 106, and blood exhaust apertures 117. The catheter 112 may be connected to the inlet area 110 of the cannula 108 in some embodiments. The inlet area 110 may be located near the proximal end 105 of the cannula, and the outlet area 106 may be located toward the distal end 107 of the cannula 108. The inlet area 110 may include a pump housing 103 with a peripheral wall 111 extending about a rotation axis of the impeller blades, positioned radially outward of the inner surface with respect to the rotation axis of the impeller. The impeller may be rotatably coupled to the pump 101 at the inlet area 110 adjacent to the blood exhaust apertures 117 formed in the peripheral wall 111 of the pump housing 103. The pump housing 103 may be composed of a metal in accordance with some implementations. The atraumatic extension 102, also referred to as a “pigtail,” may be connected to the distal end 107 of the cannula 108 and may assist with stabilizing and/or positioning the blood pump assembly 100 into the correct position in the heart. The atraumatic extension 102 may be configurable from a straight to a partially curved configuration. The atraumatic extension 102 may be composed, at least in part of a flexible material, and may have dual stiffness. It should be appreciated that some embodiments of the pump assembly may not include atraumatic extension 102.

The cannula 108 may have a shape which matches (or is similar to) the anatomy of the right ventricle of a patient. In the exemplary embodiment shown in FIG. 1A, the cannula has a proximal end 105 arranged to be located near the patient's inferior vena cava, and a distal end 107 arranged to be located near the pulmonary artery. The cannula 108 may include a first segment S1 extending from the inflow area to a point B between the inlet area 110 and the outlet area 106. The cannula 108 may also include a second segment S2 extending from a point C, which is between the inlet area 110 and the outlet area 106, to the outlet area 106. In some implementations, points B and C may be located at the same location along cannula 108. The first segment S1 of the cannula may form an ‘S’ shape in a first plane. In some implementations, segment S1 can have curvatures between 30 degrees and 180 degrees. The second segment S2 of the cannula may form an ‘S’ shape in a second plane. In some implementations, segment S2 can have curvatures between 30 degrees and 180 degrees (e.g., 40°, 50°, 60°, 70°, 80°, 90°, 100°, 110°, 120°, 130°, 140°, 150°, 160°, or 170°). The second plane can be different from the first plane. In some implementations, the second plane may be parallel or identical to the first plane.

Although shown with an ‘S’ shape, it will be appreciated that other implementations of the blood pump assembly may be formed with other shapes (e.g., a ‘U’ shape), or with no shape at all when outside the body. In such implementations, the cannula may be formed of a flexible material such that the cannula may bend during insertion and achieved the desired shape once inside the heart of the patient.

In some implementations, the blood pump assembly 100 may be inserted percutaneously through the internal jugular vein, though the right atrium and into the right ventricle. When properly positioned, the blood pump assembly 100 may deliver blood from the inlet area 110, which sits inside the patient's right atrium, through the cannula 108, to the blood exhaust apertures 117 of the pump housing 103 positioned in the pulmonary artery. Alternatively, in some implementations the blood pump assembly 100 may be inserted percutaneously through the femoral artery and into the left ventricle to deliver blood from the left ventricle into the aorta.

FIG. 1B shows that blood pump assembly 100 may form part of a cardiac support system 120. Cardiac support system 120 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of blood pump assembly 100. During operation, purge subsystem 150 may be configured to deliver a purge fluid to blood pump assembly 100 through catheter 112 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between blood pump assembly 100 and controller 130. Plug 170 may connect catheter 112, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition blood pump assembly 100 in the patient's heart (e.g., by holding a position of the pump assembly relative to the patient).

As shown in FIG. 1B, in some embodiments, the cardiac support system 120 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 120 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to blood pump assembly 100. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 may include a flexible rubber diaphragm that provides the additional filling volume by means of an expansion chamber. Infusion filter 158 may help prevent bacterial contamination and air from entering catheter 112. Sidearm 159 may provide a fluidic connection between infusion filter 158 and plug 170. Although shown as having separate purge tubing and connector cable, it will be appreciated that in some embodiments, the cardiac support system 120 may include a single connector with both fluidic and electric lines connectable to the controller 130.

During operation, controller 130 may be configured to receive measurements from one or more pressure sensors (not shown) included as a portion of blood pump assembly 100 and purge disc 154. Controller 130 may also be configured to control operation of the motor (not shown) of the blood pump assembly 100 and purge cassette 153. In some embodiments, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter 112 and plug 170. Sensor cables (not shown) within catheter 112, connector cable 160, and plug 170 may provide an electrical connection between components of the blood pump assembly 100 (e.g., one or more pressure sensors) and controller 130. Motor cables (not shown) within catheter 112, connector cable 160, and plug 170 may provide an electrical connection between the motor of the blood pump assembly 100 and controller 130. During operation, controller 130 may be configured to receive measurements from one or more pressure sensors of the blood pump assembly 100 through the sensor cables (e.g., optical fibers) and to control the electrical power delivered to the motor of the blood pump assembly 100 through the motor cables. By controlling the power delivered to the motor of the blood pump assembly 100, controller 130 may be operable to control the speed of the motor.

Various modifications can be made to cardiac support system 120 and one or more of its components. For instance, one or more additional sensors may be added to blood pump assembly 100. In another example, a signal generator may be added to blood pump assembly 100 to generate a signal indicative of the rotational speed of the motor of the blood pump assembly 100. As another example, one or more components of cardiac support system 120 may be separated. For instance, display 140 may be incorporated into another device in communication with controller 130 (e.g., wirelessly or through one or more electrical cables).

As described herein, a heart pump (e.g., blood pump assembly 100) may include a pressure sensor (e.g., an optical pressure sensor) configured to detect a pressure near an outlet of the heart pump where blood is expelled. For instance, when a right heart cardiac support device is positioned properly, the outlet of the heart pump may be positioned within the pulmonary artery of a patient's heart, and the pressure sensor may measure the pressure within the pulmonary artery. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate of blood through the heart pump. For a right heart cardiac support device, the differential pressure between the right atrium and the pulmonary artery may also indirectly be determined based on the pressure signal measuring the pressure in the pulmonary artery and the set of stored values. Alternatively, a differential pressure between the right atrium and the pulmonary artery may be determined using multiple pressure sensors, one located at an inflow region of the heart pump and another located at an outflow region of the heart pump.

As described herein, a pressure signal sensed with a pressure sensor of a heart pump located within the right side of the heart may be weaker than a corresponding pressure signal when the heart pump is located within the left side of the heart. Accordingly, some conventional techniques for determining the positioning of a left heart cardiac support device, such as determining the position based on whether the pulsatility of the pressure signal is above/below a threshold pulsatility value, may not work well to determine the positioning of a right heart cardiac support device.

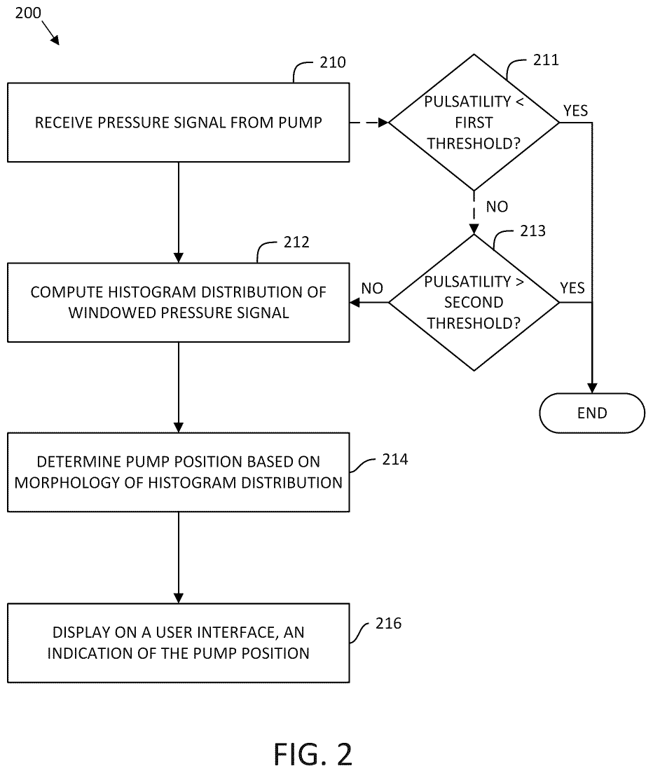

FIG. 2 illustrates a process 200 for determining a position of a heart pump in the heart of a patient based, at least in part, on a morphology of a pressure signal, in accordance with some embodiments of the present disclosure. In act 210, a pressure signal may be received from a pressure sensor of a heart pump. For example, the pressure signal may be received by a controller (e.g., controller 130) coupled to a pressure sensor of the heart pump. In some embodiments, the pressure signal may be a differential pressure signal that represents a difference in pressure between the right atrium (e.g., central venous pressure) and the pulmonary artery when the heart pump is placed properly in the right side of the heart. Alternatively, when the heart pump is not placed properly in the right side of the heart, the differential pressure signal may represent a difference in pressure between the right atrium and the right ventricle. Some embodiments of the present disclosure relate to techniques for analyzing the differential pressure signal to distinguish between a “good” placement of a heart pump (e.g., when the outlet of the heart pump is in the pulmonary artery) from a “bad” placement (e.g., when the outlet of the heart pump is in the right ventricle).

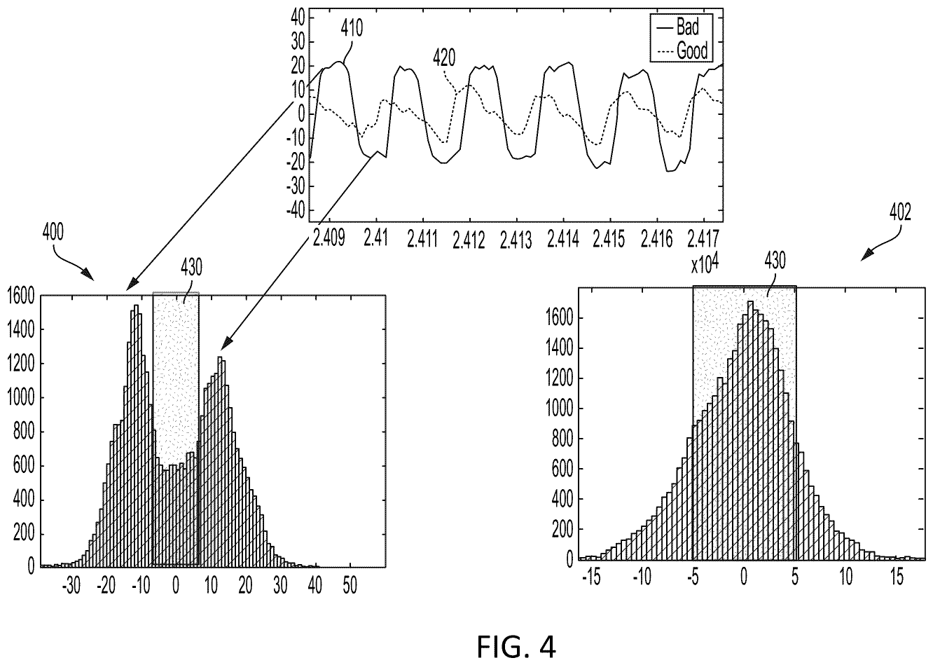

After receiving the pressure signal, process 200 may proceed to act 212, where a histogram distribution of values observed within a time widow of the received pressure signal is computed. For instance, the received pressure signal may be continuously received by a controller and values of the pressure signal within a particular time window (e.g., 5 seconds) may be used to generate a histogram of values within the time window. An example of such a histogram 400 is shown in FIG. 4. In some embodiments, pulsatility information associated with received pressure signal may be used to determine when and/or whether to compute a histogram distribution of values in act 212. For instance, as shown in FIG. 2, a pulsatility of the pressure signal within a time window may be determined, and in act 211 when it is determined that the pulsatility is less than a first threshold pulsatility value, it may be determined not to calculate the histogram distribution in act 212 and process 200 may end. In some embodiments, the pulsatility of the pressure signal may be compared to a second threshold pulsatility value, and if the pulsatility associated with the pressure signal is above the second threshold pulsatility value in act 213, it may be an indication that the patient is moving, and it may be determined not to calculate the histogram distribution in act 212 (e.g., because the patient may be moving) and process 200 may end. In some embodiments, if the pulsatility associated with the pressure signal is between the first threshold pulsatility value and the second threshold pulsatility value, it may be determined to calculate the histogram distribution in act 212 and process 200 may continue as described herein.

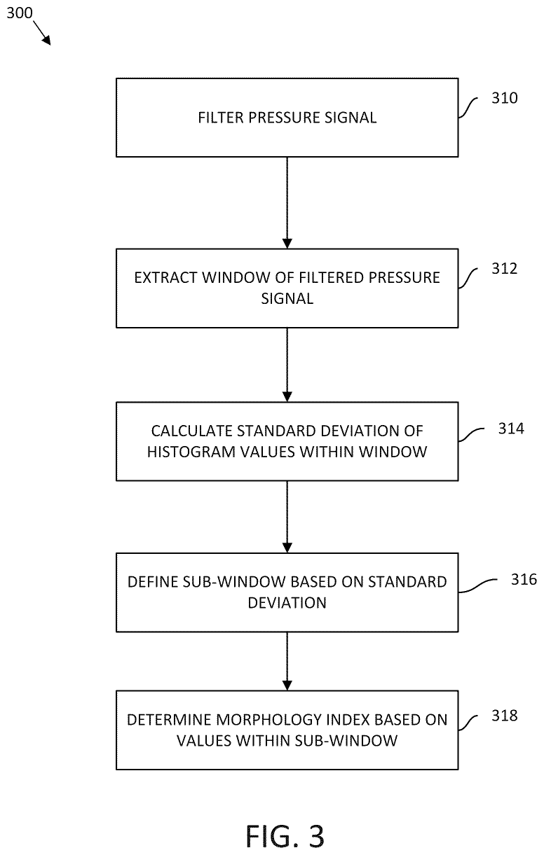

Process 200 may then proceed to act 214, where the position of the heart pump may be determined based, at least in part, on a morphology of the histogram distribution. For instance, in some embodiments, the position of the heart pump may be determined based, at least in part, on whether the histogram has a bimodal distribution or some other distribution. As described in further detail below, when the histogram has a bimodal distribution, it may be determined that the position of the pump is not positioned properly (e.g., the outlet of the pump is located in the right ventricle rather than the pulmonary artery). Examples of determining the position of a pump using morphology information associated with the histogram distribution of values generated from a pressure signal is described in more detail in connection with FIGS. 3 and 4.

Process 200 may then proceed to act 216, where an indication of the pump position is displayed on a user interface (e.g., on a display associated with controller 130). The indication of the pump position may be displayed in any suitable way. For example, an alarm or other alert may be displayed on the user interface when a “bad” position of the heart pump is determined, which may indicate to the user that the position of the heart pump should be adjusted. As another example, the display may have color indicators (e.g., green and red, to indicate “good” and “bad” positioning of the device, respectively).

FIG. 3 is a flowchart of a process 300 for determining a morphology index associated with a pressure signal from a heart pump, in accordance with some embodiments of the present disclosure. The morphology index may be used to determine the positioning of the heart pump with the heart of a patient. Process 300 may begin in act 310, where a pressure signal (e.g., a differential pressure signal received from a pressure sensor of heart pump) is filtered. In some embodiments, a high-pass filter may be used to remove the DC value and low-frequency components of the pressure signal that may represent the patient breathing and/or sensor drift. In some embodiments, a band-pass filter may be used to filter the pressure signal by reducing high frequency fluctuations in the signal while preserving the overall morphology of the signal. In some embodiments, the filter may be a linear phase filter (e.g., a finite impulse response (FIR) filter). Process 300 may then proceed to act 312, where a time window of values of the filtered pressure signal may be extracted for further analysis. In some embodiments, a time window of fixed length may be used. In some embodiments, the length of the time window may be selected based, at least in part, on one or more criteria including, but not limited to, a specificity criterion and/or an alarm updating criterion. For instance, if the length of the time window is selected to be too short, not enough of the signal may be captured within the window to distinguish good and bad placements (i.e., the values in the windowed signal may not have sufficient specificity). Alternatively, if the length of the time window is selected to be too long, the amount of time between alarm updates (e.g., when a bad placement is detected) may be too long. In some embodiments, the length of the time window is selected to be between four and ten seconds. In some embodiments, the length of the time window is selected to be between five and seven seconds. In some embodiments, the length of the time window is selected to be six seconds.

Process 300 may then proceed to act 314, where a standard deviation of histogram values within the time window is calculated. Process 300 may then proceed to act 316, where a sub-window within the time window is defined based, at least in part, on the calculated standard deviation of the histogram values in the time window. For instance, the sub-window may be centered within the time window and may have a width that is a multiple of the standard deviation. In some embodiments, the width of the time window may be half a standard deviation (e.g., +/−0.25 SD), one standard deviation (e.g., +/−0.5 SD), two standard deviations (i.e., +/−1 SD), or some other multiple of the standard deviation calculated in act 314. Process 300 may then proceed to act 318, where a morphology index (MI) value associated with the pressure signal may be determined based, at least in part, on the values of the histogram within the sub-window. For instance, in some embodiments, the MI value associated with the pressure signal may be determined as the sum of all histogram values in the sub-window divided by the sum of all histogram values in the entire time window. The MI value associated with the pressure signal may then be used to determine the pump position, as described in connection with process 200. For instance, the MI value may be compared to a threshold value, and when the MI value is less than a threshold value, it may be determined that the histogram distribution is bimodal. In such an instance, the pump may be determined not to be positioned properly (e.g., the pump output is located in the right ventricle rather than the pulmonary artery). By contrast, when the MI value is greater than the threshold value, it may be determined that the pump is positioned properly (e.g., the output of the heart pump may be located in the pulmonary artery).

FIG. 4 schematically illustrates how different pressure signals received from a heart pump pressure sensor may map to different histogram distributions, in accordance with some embodiments. FIG. 4 shows two different exemplary pressure signals. A first pressure signal 410 represents a “bad” placement of the heart pump, in which the outlet of the heart pump is located in the right ventricle of the patient's heart. As shown, when the output of the heart pump is not properly placed in the pulmonary artery, the differential pressure signal spends most of the time either up or down with a sharp slope between the peaks and troughs of the signal. The sharper peaks and troughs of the first pressure signal 410 are represented as two corresponding peaks in the histogram 400 generated from a windowed version (e.g., a six second window) of the first pressure signal 410. In other words, a bimodal histogram is generated when there is “bad” placement signal.

A second pressure signal 420 represents a “good” placement of the heart pump, in which the outlet of the heart pump is located in the pulmonary artery to provide right heart support. As shown, when the output of the heart pump is properly placed in the pulmonary artery, the differential pressure signal spends about the same amount of time up and down, with a shallower slope between the peaks and troughs in the signal. The broader peaks and troughs in the second pressure signal 420 result in a more normally-distributed histogram 402 with a single peak having a value close to zero.

As described in connection with process 300 of FIG. 3, a technique for distinguishing between a bimodal distribution (e.g., histogram 400) from a normal distribution (e.g., histogram 402) in accordance with some embodiments, may be to define a sub-window 430 that includes a central portion of the histogram and observing the presence or absence of a peak in that sub-window (e.g., the presence or absence of a peak near zero). The width of the sub-window 430 may be determined in some embodiments, based on a standard deviation of values in the histogram of the windowed pressure signal. In the example histograms shown in FIG. 4, the width of sub-window 430 is defined as one standard deviation (+/−0.5 SD).

Although a specific technique of generating a morphology index based on histogram values in a sub-window to determine whether the histogram has a bimodal distribution is described herein, it should be appreciated that other techniques for determining whether the distribution of a histogram has a bimodal distribution may alternatively be used. For example, a peak detection technique may be used to determine whether the histogram distribution has one or two peaks. In such an example, an alternative sub-window (or windows) may be chosen, with the process configured to look for the existence (or intensity) or a peak in that window or windows. In another such example, a slope of the histogram values may be used to determine one or more local maxima representing one or more peaks in the histogram. As will be appreciated, other suitable techniques for evaluating the histograms may be used in other embodiments to determine whether a bimodal or normal histogram has been generated.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/of” should be construed in the same fashion, i.e., “one or more” of the elements so conjoined. Other elements may optionally be present other than the elements specifically identified by the “and/of” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of determining a position of a heart pump in a heart of a patient, the method comprising:
receiving a pressure signal from at least one pressure sensor arranged on the heart pump;
generating a histogram of values observed within a time window associated with the pressure signal; and
determining the position of the heart pump based, at least in part, on a morphology of the histogram.

2. The method of claim 1, wherein the at least one pressure sensor comprises a differential pressure sensor.

3. The method of claim 1, wherein a length of the time window is at least four seconds and less than ten seconds.

4. The method of claim 3, wherein the length of the time window is at least five seconds and less than seven seconds.

5. The method of claim 1, further comprising:
filtering the pressure signal to generate a filtered pressure signal,
wherein generating a histogram of values comprises generating a histogram of values observed within a time window of the filtered pressure signal.

6. The method of claim 5, wherein filtering the pressure signal comprises filtering the pressure signal with a finite impulse response (FIR) filter.

7. The method of claim 1, wherein determining the position of the heart pump based, at least in part, on a morphology of the histogram comprises determining the position of the heart pump based, at least in part, on a distribution of the histogram.

8. The method of claim 7, wherein determining the position of the heart pump based, at least in part, on a distribution of the histogram comprises determining whether the distribution is a bimodal distribution.

9. The method of claim 8, wherein determining the position of the heart pump based, at least in part, on a distribution of the histogram further comprises:
determining that the heart pump is not positioned properly when the histogram has the bimodal distribution.

10. The method of claim 1, wherein determining the position of the heart pump based, at least in part, on a distribution of the histogram further comprises determining whether the distribution is a normal distribution.

11. The method of claim 10, wherein determining the position of the heart pump based, at least in part, on a distribution of the histogram further comprises:
determining that the heart pump is positioned properly when the histogram has the normal distribution.

12. The method of claim 1, wherein determining the position of the heart pump based, at least in part, on a morphology of the histogram comprises:
determining a standard deviation of values within the time window;
defining a sub-window within the time window based, at least in part, on the standard deviation; and
determining the position of the heart pump based, at least in part, on the values within the sub-window.

13. The method of claim 12, wherein determining the position of the heart pump based, at least in part, on the values in the sub-window comprises:
calculating a first sum of all values within the sub-window;
calculating a second sum of all values within the time window;
dividing the first sum by the second sum to determine a morphology index value;
determining that the heart pump is not positioned properly when the morphology index value is less than a threshold value; and
determining that the heart pump is positioned properly when the morphology index value is greater than the threshold value.

14. The method of claim 1, further comprising:
outputting via a user interface, an indication of the position of the heart pump.

15. The method of claim 1, further comprising:
determining a pulsatility of the pressure signal,
wherein generating a histogram of values is performed in response to the pulsatility of the pressure signal being above a first threshold pulsatility value and below a second threshold pulsatility value.

16. The method of claim 1, wherein
the heart pump is configured to provide right heart support for the patient, and
determining the position of the heart pump based, at least in part, on a morphology of the histogram comprises determining whether an outlet of the heart pump is located in a pulmonary artery of the patient.

17. A heart pump system, comprising:
a heart pump including at least one pressure sensor configured to sense a pressure within a portion of a heart of a patient; and
a controller configured to:
receive a pressure signal output from the at least one pressure sensor;
generate a histogram of values observed within a time window associated with the pressure signal; and
determine a position of the heart pump based, at least in part, on a morphology of the histogram.

18. The heart pump system of claim 17, wherein the at least one pressure sensor comprises a differential pressure sensor.

19-32. (canceled)

33. A controller for a heart pump system, the controller comprising:
at least one hardware processor configured to:
receive a pressure signal output from at least one pressure sensor arranged on a heart pump of the heart pump system;
generate a histogram of values observed within a time window associated with the pressure signal; and
determine a position of the heart pump based, at least in part, on a morphology of the histogram.

34-38. (canceled)

39. The controller of claim 33, wherein determining the position of the heart pump based, at least in part, on a morphology of the histogram comprises determining the position of the heart pump based, at least in part, on a distribution of the histogram.

40-57. (canceled)
